(19) United States
(12) Patent Application Publication
(10) Pub. No.: US 2012/0116354 A1
(43) Pub. Date: May 10, 2012

(54) DEVICES AND METHODS FOR SMALL VESSEL ACCESS

(76) Inventor: Richard R. Heuser, Phoenix, AZ (US)

(21) Appl. No.: 12/941,952

(22) Filed: Nov. 8, 2010

(51) Int. Cl. A61M 25/00

(52) U.S. Cl. 604/528; 606/191

(57) ABSTRACT

Disclosed herein are methods and devices for small-vessel access to the vasculature for vascular and cardiac procedures such as diagnostics and interventions, particularly methods and devices for radial, brachial, popliteal, pedal, carotid and/or axillary access to the vasculature. These methods and devices permit vascular and cardiac procedures to be carried out through small vessels, such as the radial or brachial arteries, with a reduced number of steps for the physician and reduced pain and trauma for the patient. As such, the devices and methods may improve a number of outcomes for the patient, such as by reducing the risk of bleeding complications and increasing the speed with which the patient resumes ambulation and other activities following the procedure.

### CROSS REFERENCE TO RELATED APPLICATIONS

The present application is related to U.S. patent application Ser. No. 12/140,183, filed Jan. 8, 2009, entitled “CATHETER GUIDEWIRE SYSTEM USING CONCENTRIC WIRES;” and U.S. Pat. No. 7,402,141, issued Jul. 22, 2008, entitled “CATHETER GUIDEWIRE SYSTEM USING CONCENTRIC WIRES,” the disclosures of which are hereby incorporated by reference in their entirety.

### TECHNICAL FIELD

Embodiments relate to methods and devices for small-vessel access to the vasculature for vascular and cardiac procedures such as diagnostics and interventions, particularly methods and devices for radial, brachial, and/or axillary access to the vasculature.

### BACKGROUND

Radial artery access for percutaneous vascular and cardiac interventions and diagnostics has been shown to reduce complications when compared to the standard femoral artery approach. For example, interventions accomplished via the radial artery carry a lower risk of bleeding complications and a higher rate of early ambulation. However, such an approach is complicated and requires a number of steps in order to insert a sheath of sufficient size to carry out the interventions or diagnostics.

The technique first requires a local anesthetic to be administered to the wrist with a small needle. However, swelling from the local anesthetic often makes it difficult to detect the radial pulse and causes pain for the patient. Next, a micro puncture system is used to puncture the radial artery. Blood returns to the small micro puncture needle, and a very small wire (e.g., approximately 0.018 inches) is passed into the vessel. Next, a 4 French micro puncture sheath is inserted, and the inner dilator is removed. The 4 French sheath is large enough to pass a 0.035 inch wire, but is not big enough to pass catheters, so a larger wire such as a 0.035 inch wire is inserted, the 4 French sheath is removed, and a larger sheath, such as a 5 French or 6 French sheath, is inserted in place of the smaller sheath. Although this procedure typically has a better outcome compared to a traditional femoral approach, the multiple steps required for this procedure cause pain and trauma for the patient and increase the complexity and expense of the procedure.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates a cross-sectional side view of an embodiment of a guidewire system as disclosed herein, showing three concentric wires, including the proximal and distal ends, central lumens, and proximal handles, in accordance with various embodiments;

FIG. 2 illustrates an embodiment of the method in which a needle (for example, a 24 gauge hollow needle) may be placed in the left radial artery, and a first or inner wire, such as a 0.014 inch wire, may be positioned therethrough, in accordance with various embodiments;

FIG. 3 illustrates an embodiment of the method in which a second wire, such as a steel alloy 0.018 wire, may be passed over the first or inner wire, dilating the skin and arteriotomy site as it advances, in accordance with various embodiments;

FIG. 4 illustrates an embodiment of the method in which a sheath-in-sheath device may be advanced over the second wire, further dilating the skin aperture and arteriotomy site and allowing the passage of a 0.035 inch wire and a 6 French or smaller sheath once the two dilators are removed, in accordance with various embodiments;

FIGS. 5A-C illustrate examples of concentric wire devices, including a wire-on-wire device (FIG. 5A), a screw-on concentric wire device (FIG. 5B), and a snap-on concentric wire device (FIG. 5C), in accordance with various embodiments;

FIG. 6 is a flow chart illustrating an example of a method of using a concentric wire device for small vessel access, in accordance with various embodiments; and

FIG. 7 is a flow chart illustrating another example of a method of using a concentric wire device for small vessel access, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide methods and devices for small-vessel access to the vasculature that reduce the amount of equipment, the number of steps, and/or the amount of trauma to the subject involved in a typical radial access procedure. In various embodiments, the disclosed methods and devices may be used for any arterial approach, including a traditional femoral artery approach, but the disclosed methods and devices may be particularly well-suited for use in smaller vessels, including a radial approach, brachial approach, or axillary approach, or the antegrade stick approach, popliteal stick approach or pedal approach, for example in subjects with limb ischemia, which would include the perineal vessel, the dorsalis pedis, or the anterior tibial vessel. The disclosed devices and methods also may be used for a carotid application with a percutaneous puncture in the neck.

The main limitation on catheter placements for percutaneous interventions and diagnostics in smaller vessels, including the radial and foot arteries, is the bulkiness of current sheaths, wires, and dilators. In various embodiments, of the methods disclosed herein, instead of beginning the procedure with an injection of local anesthetic, a small, hollow needle, such as a needle that normally might be used to administer the anesthetic, is passed into the artery and used to position a thin wire. A second hollow wire may then be passed over the first wire. The second wire may be tapered such that advancement of the second wire gently dilates the vessel without causing trauma. Optionally, for example if further dilation is desired, a third hollow wire may then be passed over the second wire. The third wire also may be tapered such that advancement of the third wire gently dilates the vessel without causing trauma. Finally, in some embodiments, a standard sheath may be advanced along the wires, and optionally, the first and/or second wires may be removed, leaving the second or third wire in place.

In some embodiments, rather than advancing a second hollow, tapered wire along the first wire, the second wire may be coupled to the proximal end of the first wire, for example either with a screw-on type connection or a snap-on type connection. In various embodiments, the junction between the first and second wires may be very smooth and gently tapered so as to prevent trauma during the dilation procedure. Optionally, a third wire may be coupled to the second wire in a similar fashion, creating a three-part dilating wire.

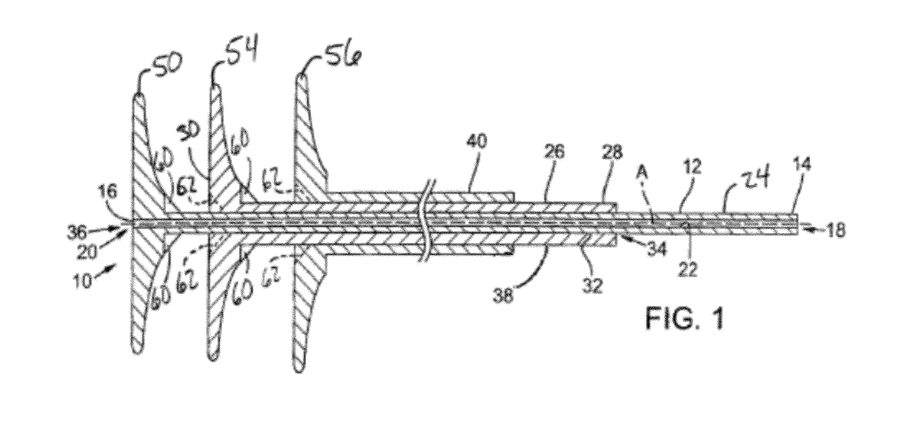

Various embodiments of the methods may make use of a concentric wire catheter guidewire system such as disclosed in U.S. Pat. No. 7,402,141. For example, as described above, a system of two or more concentric wires may be used to dilate the vessel. As shown in FIG. 1, an embodiment of the concentric wire system used to carry out the methods disclosed herein may be a multiple concentric wire system, indicated generally at 10. In various embodiments, system 10 may include an inner wire 12 having a distal end 14 and a proximal end 16.

A first or inner wire 12 may have a length that may be selected for a particular type of procedure to be conducted in a human blood vessel, e.g., between about 30 cm and about 100 cm, for example about 60 cm. Inner wire 12 may include an opening 18 adjacent distal end 14 and an opening 20 adjacent proximal end 16, and a central lumen 22 extending between the proximal and distal openings. In various embodiments, central lumen 22 may define an inner diameter for wire 12, and wire 12 also may have a generally cylindrical outer surface 24 defining an outer diameter. Typically, the maximum outer diameter of inner wire 12 may be between about 0.010 and 0.018 inches, and may be any size therebetween, or larger or smaller as selected for the desired procedure and for compatibility with other wires, catheters, sheaths, and other equipment. For example, the maximum outer diameter of inner wire 12 may be 0.010, 0.014. or 0.018 inches in specific, non-limiting examples. Although distal end 14 is depicted as squared-off, in some embodiments it may have a gradual taper, for example culminating in a point or a rounded end.

Optionally, as shown, inner wire 12 may be provided with a handle 50, which may be removable adjacent proximal end 16, so that it may be used by the physician in manipulating the wire about and along a central axis A of the wire. However, in other embodiments no handle may be included. In some embodiments, rigidity may be controlled by the use of braiding or the selection of various materials. For example, nitinol may be used for flexibility, but it may be made stiffer by adding more stainless steel. In some embodiments, inner wire 12 may be comprised substantially completely of stainless steel. In some embodiments, no hydrophilic coating may be applied to the wires. Without being bound by theory, it is believed that wires that are not hydrophilic may pass through the skin and/or artery more easily than hydrophilic wires do.

A second wire 26, which may be constructed to be deployed over inner wire 12, may include a distal end 28 and a proximal end 30 and a length preferably selected to be compatible with inner wire 12. In various embodiments, a central lumen 32 of wire 26 may extend between a distal opening 34 and a proximal opening 36. Central lumen 32 of second wire 26 may define an inner diameter for the wire, and second wire 26 may have a generally cylindrical outer surface 38 defining an outer diameter. In various embodiments, the maximum outer diameter of second wire 26 may be between about 0.016 and about 0.035 inches, for example about 0.018 inches, about 0.023 inches, or about 0.035 inches in specific, non-limiting examples, and may be any size therebetween, or larger or smaller as selected for the desired procedure and for compatibility with other wires, catheters, sheaths, and other equipment. Although distal end 28 is depicted as squared-off, in some embodiments it may have a gradual taper, for example culminating in a point or a rounded end.

Optionally, second wire 26 may be provided with a handle 54, which may be removable, adjacent proximal end 30 that the physician may use in manipulating the wire about and along a central axis A of the wire. However, in other embodiments no handle may be included. In some embodiments, second wire 26 may be comprised substantially completely of stainless steel.

In some embodiments, such as the depicted embodiment, system 10 may also include a third or outer wire 40 having proximal and distal ends with openings and a central lumen communicating therebetween, inner and outer diameters, and a generally cylindrical outer surface as for the other wires. In some embodiments, third wire 40 may be sized to fit over second wire 26, and optionally may include a handle 56 that may be removably coupled adjacent the proximal end for manipulation of the third wire about and along central axis A. Third wire 40 may have an outer diameter of between about 0.030 inches and about 0.040 inches, for example about 0.035 inches, and may be any size therebetween, or larger or smaller as selected for the desired procedure and for compatibility with other wires, catheters, sheaths, and other equipment. Typically, the length of third wire 40 may be less than the length of second wire 26, and the length of second wire 26 may be less than that of inner wire 12.

In one specific, non-limiting example of a suitable concentric wire system, inner wire 12 may have an outer diameter of about 0.010, 0.012, or 0.014 inches, second wire 26 may have an outer diameter of about 0.018 or 0.021 inches, and third wire 40 may have an outer diameter of about 0.035 inches. In various embodiments, such a concentric wire system may be compatible with a 4 French catheter system, 5 French catheter system, or a 6 French catheter system. In another specific, non-limiting example, inner wire 12 may have an outer diameter of about 0.010, 0.012, or 0.014 inches, second wire 26 may have an outer diameter of about 0.035 inches, and no third wire may be needed.

In various embodiments, the length of inner wire 12 may be between about 50 cm and about 70 cm, for example about 60 cm, but may be other sizes as desired for particular procedures. Typically, the length of second wire 26 may be about 5-10 cm shorter than inner wire 12, and the length of third wire 40 may be about 5-10 cm shorter than second wire 26. In one specific, non-limiting example, the length of inner wire 12 may be about 60 cm, the length of second wire 26 may be about 50 cm, and the length of third wire 40 may be between about 40 cm.

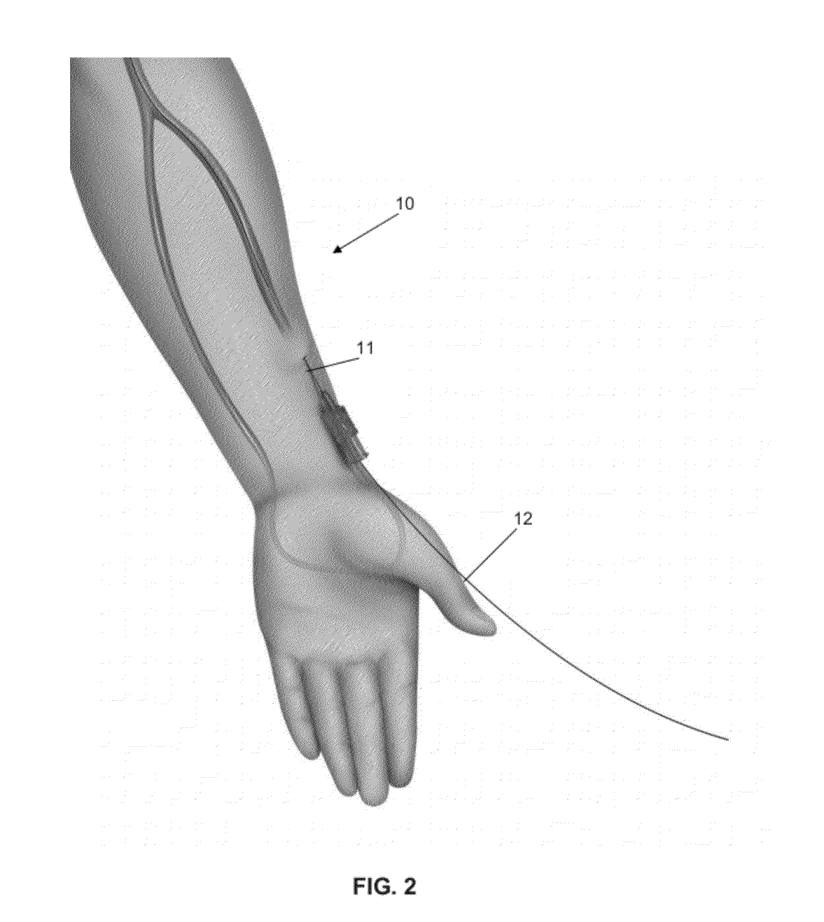

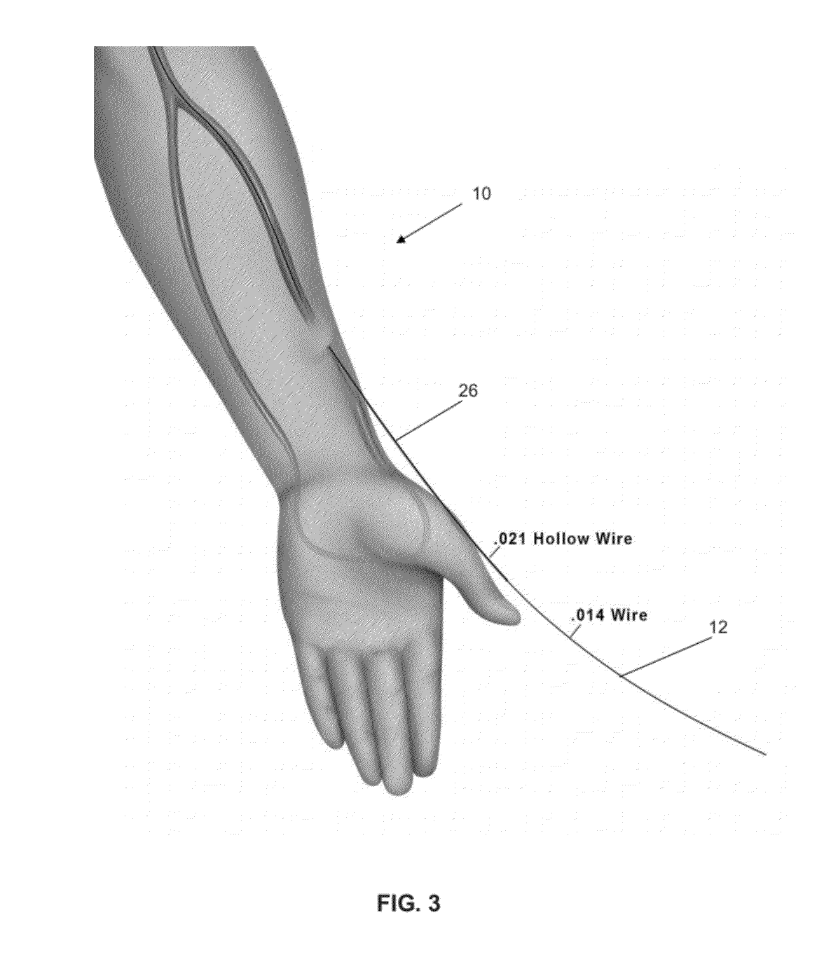

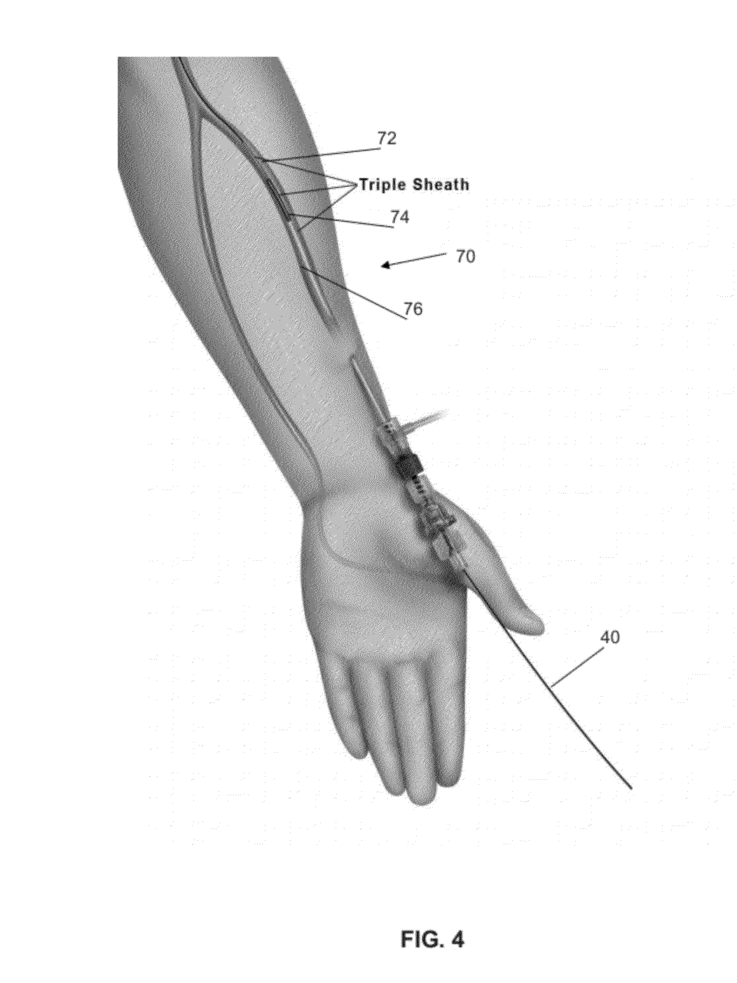

FIG. 2 illustrates an embodiment of the method in which a needle (for example, a 24 gauge hollow needle) is placed in the left radial artery and a first or inner wire, such as a 0.014 inch wire, is positioned therethrough, in accordance with various embodiments; FIG. 3 illustrates an embodiment of the method in which a second wire, such as a steel alloy 0.018 wire, is passed over the first or inner wire, dilating the skin and arteriotomy site as it advances, in accordance with various embodiments; and FIG. 4 illustrates an embodiment of the method in which a sheath-in-sheath device is advanced over the second wire, further dilating the skin aperture and arteriotomy site and allowing the passage of a 0.035 inch wire and a 6 French or smaller sheath once the two dilators are removed, in accordance with various embodiments.

As illustrated in FIGS. 2 and 3, in various embodiments, system 10 may be positioned in a desired small vessel, such as the radial artery, through the lumen of a small gauge needle 11, such as a 21 gauge, 22 gauge, 23 gauge, 24 gauge needle, or the like, or larger or smaller needles as selected for the desired procedure and for compatibility with other wires, catheters, sheaths, and other equipment. In various embodiments, once inner wire 12 has been positioned in the vessel, small gauge needle 11 may be withdrawn. In some embodiments, a local anesthetic may then be administered in order to reduce discomfort for the subject during the remainder of the procedure. Optionally, once a local anesthetic has been administered, the aperture of the hole through which wire 12 passes may be enlarged, for example with a scalpel.

As shown in FIG. 3, once wire 12 is in place, second wire 26 may be advanced along inner wire 12. Because second wire 26 may have a gradual taper, advancing second wire 26 gradually and gently dilates the aperture in the skin and vessel. In some embodiments, second wire 26 may sufficiently dilate the aperture in the skin and artery such that a sheath may be passed, for example, in instances where second wire 26 tapers to a diameter of about 0.035 inches. In other embodiments, third wire 40 may be advanced along second wire 26, further dilating the aperture in the skin and vessel. Once the aperture in the skin and vessel is sufficiently dilated, a sheath, such as a 4 French, 5 French, or 6 French sheath, may be passed over system 10, and the diagnostic or intervention procedure may be carried out.

In various embodiments, using a concentric wire system 10 to dilate the aperture in the skin and vessel may allow the procedure to be carried out more safely, more easily, and with less pain for the subject, without requiring a number of exchanges for sheaths. In some embodiments, using a set of concentric wires for dilation may the likelihood of kinking, which can occur with plastic sheaths. Additionally, the system reduces the cost of the procedure, since multiple sheaths require more surgical time and more expense. In addition, dilation with stainless steel wires is more comfortable than dilation with plastic sheaths.

Alternately, as shown in FIG. 4, once second wire 26 has been advanced, a sheath-in-sheath device 70 comprising a first 72, second 74, and optional third 76 tapered sheath may be advanced over second wire 26, further dilating the skin aperture and arteriotomy site and allowing the passage of third wire 40 and a 6 French or smaller sheath once first and second sheaths 72, 74 (e.g., the dilating sheaths or dilators) are removed. In various embodiments, first tapered sheath 72 may be tapered to provide a smooth transition between the outer diameter of second wire 26 and first tapered sheath 72, second tapered sheath 74 may be tapered to provide a smooth transition between the outer diameter of first tapered sheath 72 and second tapered sheath 74, and third tapered sheath 76 may be tapered to provide a smooth transition between the outer diameter of second tapered sheath 74 and second tapered sheath 74.

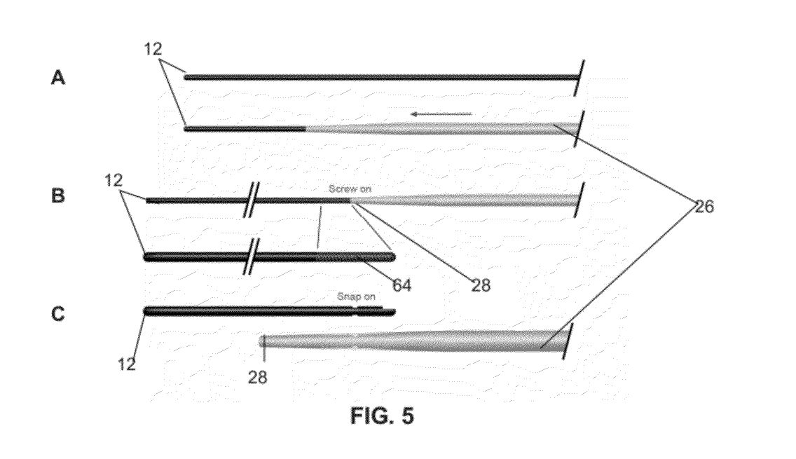

Referring to FIGS. 5A-C, as described above, in some embodiments, rather than advancing a second hollow, tapered wire along inner wire 12 (as illustrated in FIG. 2A), the second wire may be coupled to the proximal end of the inner wire, for example either with a screw-on type connection (see, e.g., FIG. 5B) or a snap-on type connector (see, e.g., FIG. 5C). In the screw-on embodiment shown in FIG. 5B, inner wire 12 may be threaded on the exterior 64 proximal end, and adapted to couple to second wire 26 via interior threads (not shown) on distal end 28. In the snap-on embodiment shown in FIG. 5C, inner wire 12 may be adapted on the proximal end to couple to second wire 26 by snapping onto distal end 28. In all three of these embodiments, the junction between the inner and second wires may be very smooth and gently tapered so as to prevent trauma during the dilation procedure. Optionally, a third wire may be coupled to the second wire in a similar fashion, creating a three-part dilating wire (not shown). In various embodiments, the two-part (or three-part) tapering wire may have an initial diameter of about 0.010, 0.012, or 0.014 inches and a final diameter of about 0.035 inches.

In use, inner wire 12 may be positioned in a vessel using a small gauge needle as discussed above, advanced, and then second wire 26 may be screwed or snapped onto the proximal end of inner wire 12. The dual wire assembly is then advanced until the vessel is sufficiently dilated, for example, to an inner diameter of about 0.035 inches, and a suitable sheath, such as a 6F sheath, is positioned as described above. The wire may then be removed or used for the procedure as desired, for example when a cocktail of antispasmodic drugs is administered.

In another embodiment, a sheath with two wire dilators may be used for small vessel access. In various embodiments, a small hollow needle may be used to position a inner wire 12 as described above, such as a 0.010, 0.012. 0.014, 0.018, or 0.021 inch wire. A sheath is placed over inner wire 12 that has a small-diameter wire dilator, such as a conventional micro-puncture dilator. In various embodiments, the micro-puncture dilator may include a second wire dilator that tapers to the diameter of a conventional 6 French sheath, allowing a 6 French sheath to be inserted. After insertion, the dilators may be removed, and the procedure may be performed vial the 6 French sheath.

In various embodiments, the two dilators may couple to one another in any of the three manners illustrated in FIG. 5 (e.g., wire-in-wire technique, screw-on, or snap-on). This procedure may save time, use a smaller needle than a conventional approach, and the wires actually dilate the skin and the artery rather than the sheath. In various embodiments, this approach may be advantageous because metal wires may be configured to tape very gradually, and this may create a less traumatic approach with less pain for the subject.

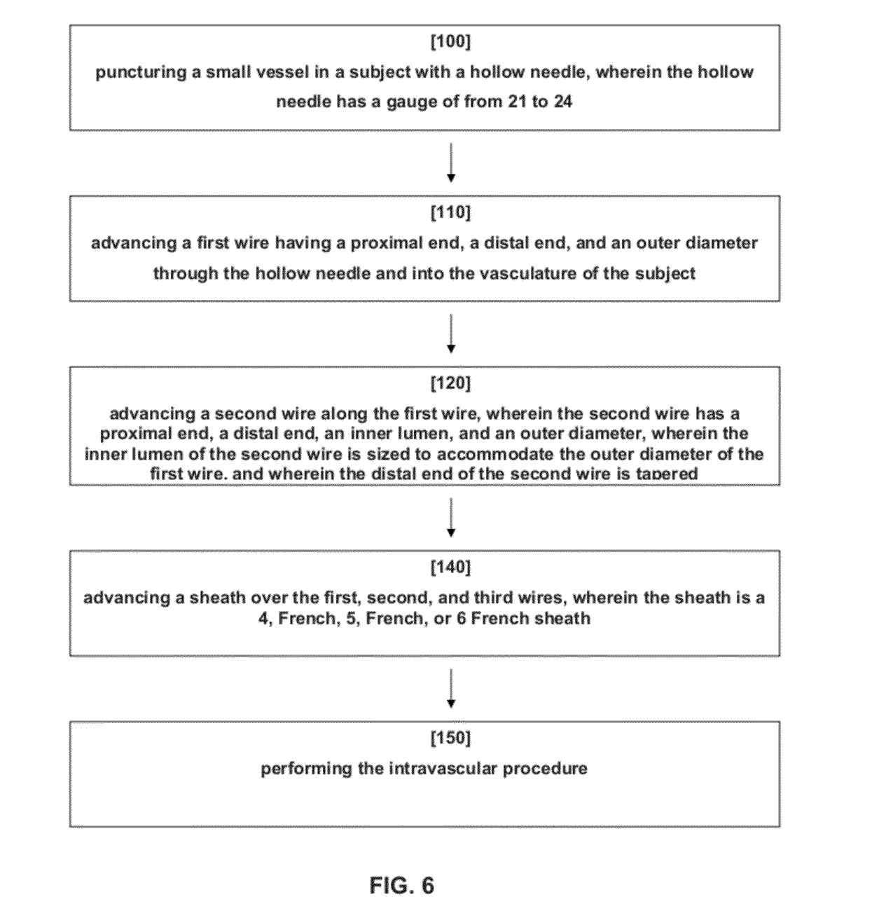

Turning now to FIG. 6, in various embodiments methods are provided for accessing a small vessel for an intravascular procedure. In the illustrated embodiment, one such method includes the steps of:

puncturing a small vessel in a subject with a hollow needle, wherein the hollow needle has a gauge of from 21 to 24;

advancing a first wire having a proximal end, a distal end, and an outer diameter through the hollow needle and into the vasculature of the subject;

advancing a second wire along the first wire, wherein the second wire has a proximal end, a distal end, an inner lumen, and an outer diameter, wherein the inner lumen of the second wire is sized to accommodate the outer diameter of the first wire, and wherein the distal end of the second wire is tapered;

advancing a sheath over the first and second wires, wherein the sheath is a 4, French, 5, French, or 6 French sheath; and

performing the intravascular procedure.

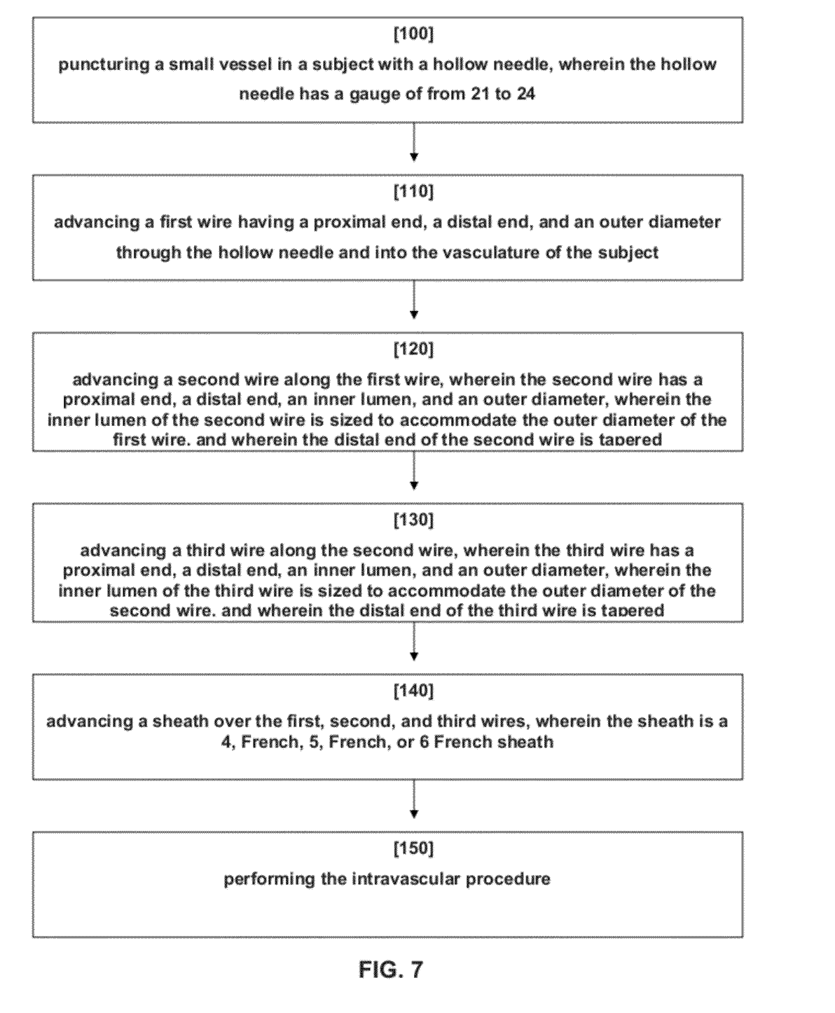

As illustrated in FIG. 7, some methods include an additional step such that the method includes:

puncturing a small vessel in a subject with a hollow needle, wherein the hollow needle has a gauge of from 21 to 24;

advancing a first wire having a proximal end, a distal end, and an outer diameter through the hollow needle and into the vasculature of the subject;

advancing a second wire along the first wire, wherein the second wire has a proximal end, a distal end, an inner lumen, and an outer diameter, wherein the inner lumen of the second wire is sized to accommodate the outer diameter of the first wire, and wherein the distal end of the second wire is tapered;

advancing a third wire along the second wire, wherein the third wire has a proximal end, a distal end, an inner lumen, and an outer diameter, wherein the inner lumen of the third wire is sized to accommodate the outer diameter of the second wire, and wherein the distal end of the third wire is tapered;

advancing a sheath over the first, second, and third wires, wherein the sheath is a 4, French, 5, French, or 6 French sheath; and

performing the intravascular procedure.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of accessing a small vessel for an intravascular procedure, comprising:
puncturing a small vessel in a subject with a hollow needle, wherein the hollow needle has a gauge of from 21 to 24;
advancing a first wire having a proximal end, a distal end, and an outer diameter through the hollow needle and into the vasculature of the subject;
advancing a second wire along the first wire, wherein the second wire has a proximal end, a distal end, an inner lumen, and an outer diameter, wherein the inner lumen of the second wire is sized to accommodate the outer diameter of the first wire, and wherein the distal end of the second wire is tapered;
advancing a sheath over the first and second wires, wherein the sheath is a 4, French, 5, French, or 6 French sheath; and
performing the intravascular procedure.

2. The method of claim 1, further comprising:
advancing a third wire along the second wire prior to advancing the sheath, wherein the third wire has a proximal end, a distal end, an inner lumen, and an outer diameter, wherein the inner lumen of the third wire is sized to accommodate the outer diameter of the second wire, and wherein the distal end of the third wire is tapered.

3. The method of claim 1, wherein the first wire has a maximum outer diameter of from about 0.010 inches to about 0.014 inches.

4. The method of claim 1, wherein the second wire has a maximum outer diameter of from about 0.018 inches to about 0.035 inches.

5. The method of claim 2, wherein the third wire has a maximum outer diameter of from about 0.030 inches to about 0.040 inches.

6. The method of claim 2, wherein the first wire has a maximum outer diameter of from about 0.010 inches to about 0.014 inches, wherein the second wire has a maximum outer diameter of from about 0.018 inches to about 0.021 inches, and wherein the third wire has a maximum outer diameter of from about 0.035 inches.

7. The method of claim 2, wherein the first, second, and third wires are formed substantially from stainless steel.

8. The method of claim 2, wherein the first, second, and third wires are substantially hydrophobic.

9. The method of claim 1, wherein the small vessel is a radial artery, brachial artery, axillary artery, popliteal artery, pedal artery or carotid artery.

10. The method of claim 1, wherein advancing the second wire into the vasculature of the subject dilates the small vessel.

11. The method of claim 2, wherein advancing the third wire into the vasculature of the subject dilates the small vessel.

12. A method of accessing a small vessel for an intravascular procedure, comprising:
puncturing a small vessel in a subject with a hollow needle, wherein the hollow needle has a gauge of from 21 to 24;
advancing a first wire having a proximal end, a distal end, and an outer diameter through the hollow needle and into the vasculature of the subject;
coupling a second wire to the first wire, wherein the second wire has a proximal end, a distal end, an inner lumen, and an outer diameter, wherein the outer diameter of the second wire is larger than the outer diameter of the first wire, and wherein the distal end of the second wire is tapered;
advancing the second wire into the vasculature of the subject;
advancing a sheath over the first and second wires, wherein the sheath is a 4, French, 5, French, or 6 French sheath; and
performing the intravascular procedure.

13. The method of claim 12, further comprising:
coupling a third wire to the second wire prior to advancing the sheath, wherein the third wire has a proximal end, a distal end, an inner lumen, and an outer diameter, wherein the outer diameter of the third wire is greater than the outer diameter of the second wire, and wherein the distal end of the third wire is tapered.

14. The method of claim 12, wherein the first wire has a maximum outer diameter of from about 0.010 inches to about 0.014 inches.

15. The method of claim 12, wherein the second wire has a maximum outer diameter of from about 0.018 inches to about 0.035 inches.

16. The method of claim 13, wherein the third wire has a maximum outer diameter of from about 0.030 inches to about 0.040 inches.

17. The method of claim 13, wherein the first wire has a maximum outer diameter of from about 0.010 inches to about 0.014 inches, wherein the second wire has a maximum outer diameter of about 0.018 inches, and wherein the third wire has a maximum outer diameter of about 0.035 inches.

18. The method of claim 13, wherein the first, second, and third wires are formed substantially from stainless steel.

19. The method of claim 13, wherein the first, second, and third wires are substantially hydrophobic.

20. The method of claim 12, wherein the small vessel is a radial artery, brachial artery, axillary artery, popliteal artery, pedal artery or carotid artery.

21. The method of claim 12, wherein advancing the second wire into the vasculature of the subject dilates the small vessel.

22. The method of claim 13, wherein advancing the third wire into the vasculature of the subject dilates the small vessel.

23. An intravascular dilation device comprising:
a first wire having a proximal end, a distal end, and a maximum outer diameter;
a second wire having a proximal end, a distal end, an inner lumen, and a maximum outer diameter, wherein the proximal end of the first wire is adapted to couple to the distal end of the second wire, wherein the maximum outer diameter of the second wire is greater than the maximum outer diameter of the first wire, and wherein coupling the second wire to the first wire creates a smooth taper between the smaller first wire and the larger second wire.

24. The intravascular dilation device of claim 23, further comprising:
a third wire having a proximal end, a distal end, and an inner lumen, and a maximum outer diameter, wherein the proximal end of the second wire is adapted to couple to the distal end of the third wire, wherein the maximum outer diameter of the third wire is greater than the maximum outer diameter of the second wire, and wherein coupling the third wire to the second wire creates a smooth taper between the smaller second wire and the larger third wire.

25. The intravascular dilation device of claim 23, wherein the first wire is coupled to the second wire via a screw-on coupling or a snap-on coupling.

26. The intravascular dilation device of claim 24, wherein the second wire is coupled to the third wire via a screw-on coupling or a snap-on coupling.

27. The intravascular dilation device of claim 23, wherein the first wire has a maximum outer diameter of from about 0.010 inches to about 0.014 inches.

28. The intravascular dilation device of claim 23, wherein the second wire has a maximum outer diameter of from about 0.018 inches to about 0.035 inches.

29. The intravascular dilation device of claim 24, wherein the third wire has a maximum outer diameter of from about 0.030 inches to about 0.040 inches.

30. The intravascular dilation device of claim 24, wherein the first wire has a maximum outer diameter of from about 0.010 inches to about 0.014 inches, wherein the second wire has a maximum outer diameter of about 0.018 inches, and wherein the third wire has a maximum outer diameter of from about 0.035 inches.

31. The intravascular dilation device of claim 23, wherein the first, second, and third wires are formed substantially from stainless steel.

32. The intravascular dilation device of claim 24, wherein the first, second, and third wires are substantially hydrophobic.
